(19) United States
(12) Patent Application Publication
(10) Pub. No.: US 2017/0328770 A1
(43) Pub. Date: Nov. 16, 2017

(54) FOOD INTEGRITY PROBE HEAD

(71) Applicant: OCEAN OPTICS, INC., Largo, FL (US)

(72) Inventors: THOMAS HOPKINS, OVIEDO, FL (US); DIETER BINGEMANN, DENKENDORF (DE); DAVID CREASEY, SAFETY HARBOR, FL (US)

(21) Appl. No.: 15/592,274

(22) Filed: May 11, 2017

Related U.S. Application Data
(60) Provisional application No. 62/335,791, filed on May 13, 2016.

(51) Int. Cl. G01J 3/02; G01N 33/02

(52) U.S. Cl. CPC G01J 3/0218; G01N 33/02

(57) ABSTRACT

A food integrity probe design comprising two illumination fiber rings, allowing for two different simultaneous measurements wherein the inner illumination ring generates a surface reflectance signal on the central detection fibers and the outer illumination ring allows for an additional interactance measurement, probing deeper into the sample than the surface reflectance alone while partially eliminating specular reflection, which reduces the signal quality, through the addition of a ring-shaped diffusor onto the inner illumination ring is disclosed.

### CROSS REFERENCE TO RELATED APPLICATION

The present application claims the benefit of previously filed co-pending Provisional Patent Application, Ser. No. 62/335,791, filed on May 13, 2016.

### FIELD OF THE INVENTION

The device of this disclosure belongs to the field of spectrometer probes. More specifically, it is a new geometry for the illumination and collection fibers of a spectrometer probe for food quality measurements.

### BACKGROUND OF THE INVENTION

Typical prior art reflection probe setups come in two geometries; two fibers, one for illumination, one for detection, or seven or eight fibers. The seven or eight fiber configurations include i) one center fiber for illumination and a six fiber outer ring for detection with the fibers arranged in line for slit on spectrometer side; ii) a ring illumination (bundle arrangement at light source), center fiber on probe for detection (this is very close to the inner probe setup in the disclosed design with the main difference being a larger number of illumination fibers in a larger diameter ring); and, iii) an outer ring illumination (seven fibers), arranged in a 30 degree angle to center probe (Hellma, Germany) such that they illuminate the point right behind the probe window.

The novel design of this disclosure is the combination of two illumination fiber rings, allowing for two different simultaneous measurements. The inner illumination ring generates a surface reflectance signal on the central detection fiber, the outer illumination ring allows for an additional interactance measurement, probing deeper into the sample than the surface reflectance alone. Also, specular reflection, which reduces the signal quality, was partially eliminated through the addition of a ring-shaped diffusor onto the (inner) illumination ring.

### BRIEF SUMMARY OF THE INVENTION

The optical device of this disclosure is an integrated optical assembly for UV-VIS-NIR-MIR reflectance, fluorescence, irradiance and transflection measurements of food stuffs. The probe features integrated illumination and collection fibers in a novel geometry for food quality measurements.

### BRIEF DESCRIPTION OF THE DRAWINGS

For a fuller understanding of the nature and objects of the invention, reference should be made to the following detailed description, taken in connection with the accompanying drawings, in which:

FIGS. 1a and 1b show perspective and front cut away views of one embodiment of the food integrity probe;

FIGS. 2a and 2b show perspective and front cut away views of a second embodiment of the food integrity probe; and,

FIG. 3 shows a front cut away view of the second embodiment of the food integrity probe that includes a liquid sample chamber.

### DESCRIPTION OF THE PREFERRED EMBODIMENT

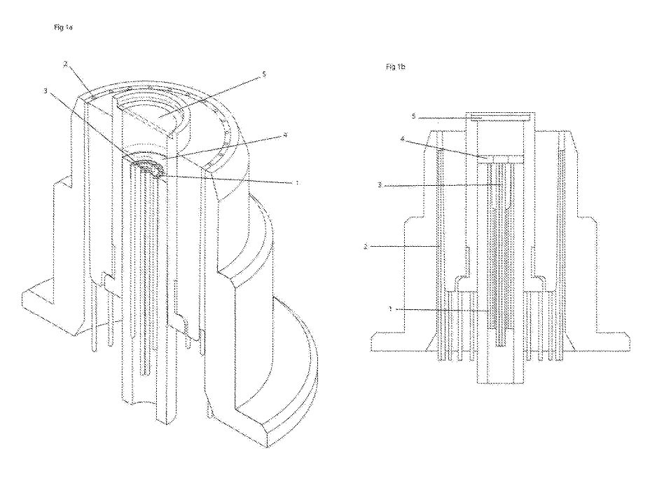

This invention is an integrated optical assembly for UV-VIS-NIR-MIR reflectance, fluorescence, irradiance and transflection measurements of food stuffs. The probe features integrated illumination and collection fibers in a novel geometry for food quality measurements as shown in the embodiments disclosed in FIGS. 1a and b and 2a and b.

As shown in both FIGS. 1a and b and 2a and b the basic geometry of the food integrity probe of this disclosure is the combination of two concentric illumination fiber rings surrounding a group of detection fibers, allowing for two different simultaneous measurements. In the preferred embodiment shown in the figures the inner illumination ring (1) of the preferred embodiments comprises 26 optical fibers and generates a surface reflectance signal on the group of 5 central detection fibers (3). The outer illumination ring (2) comprises 26 optical fibers and allows for an additional interactance measurement, probing deeper into the sample than the surface reflectance alone. The diameters as well as the numbers of these fibers can be varied based on the amount of light needed and to best suit the desired conditions as can be determined by someone skilled in the art. Also, specular reflection, which reduces the signal quality, is partially eliminated through the addition of a ring-shaped diffusor (4) onto the inner illumination ring (1). A sapphire window (5) covers the inner illumination ring (1), detection fibers (3), and ring-shaped diffusor (4) in both disclosed embodiments.

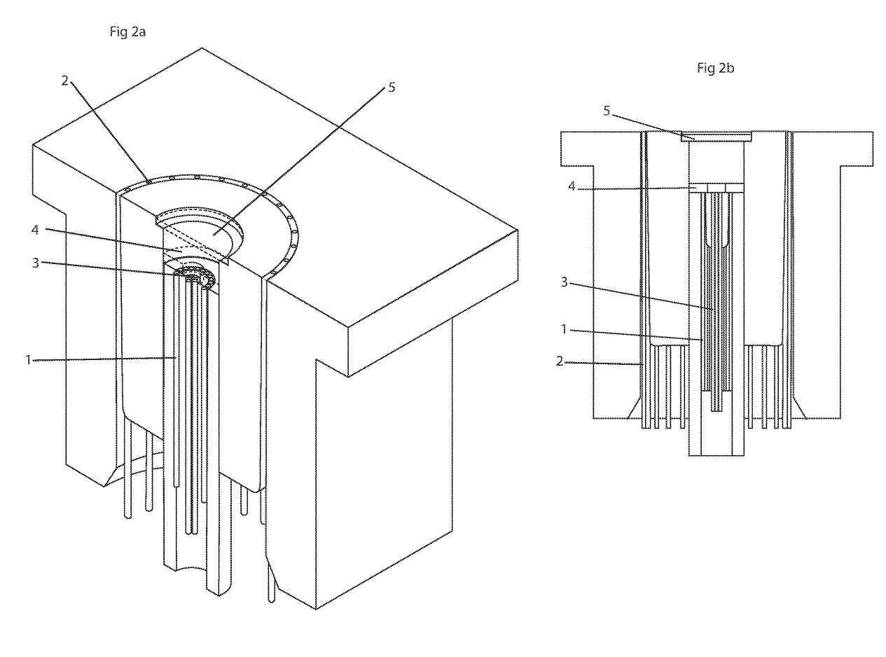

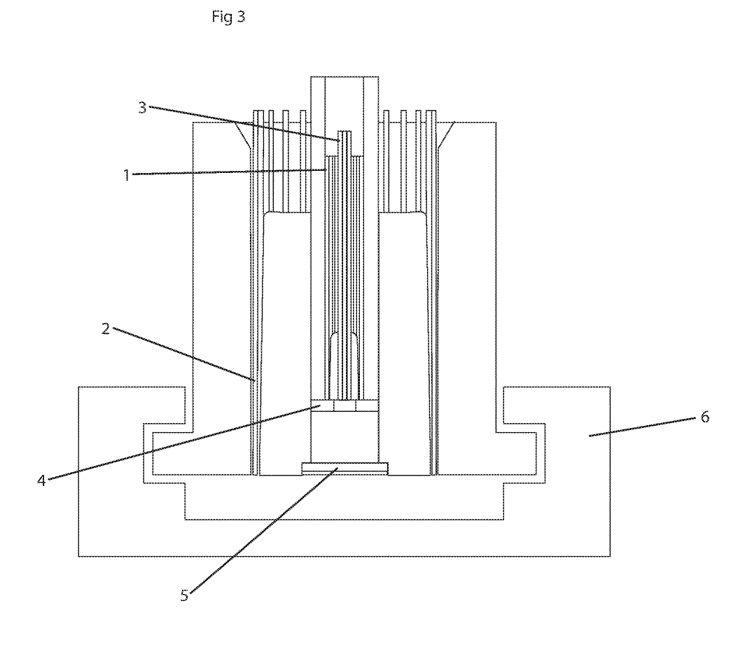

The rectangular design of the second embodiment shown in FIG. 2 of the disclosed probe enables the sliding of a sample compartment (5) for liquid samples onto the probe head to also measure liquid layers in diffuse transflectance mode as shown in FIG. 3.

Since certain changes may be made in the above described food integrity probe without departing from the scope of the invention herein involved, it is intended that all matter contained in the description thereof or shown in the accompanying figures shall be interpreted as illustrative and not in a limiting sense.

What is claimed is:

1. A spectrometer probe head for testing a food sample comprising:
a center detection group of fibers;
an inner illumination ring of fibers that when illuminated generates a surface reflectance signal from the surface of said food sample that is detected by said center detection group of fibers;
said inner illumination ring of fibers covered by a ring shaped diffuser;
a sapphire window covering said center detection group of fibers, said inner illumination ring of fibers, and said ring shaped diffuser; and,
an outer illumination ring of fibers that when illuminated generates an interactance signal from within said food sample that is detected by said center detection group of fibers.
